(19) United States
(12) Patent Application Publication
(10) Pub. No.: US 2020/0231752 A1
(43) Pub. Date: Jul. 23, 2020

(54) NOVEL BIOBASED AMINES

(71) Applicant: BASF SE, Ludwigshafen (DE)

(72) Inventors: Sophie PUTZIEN, Ampfing (DE); Olivier FLEISCHEL, Eckbolsheim (FR); Bend BRUCHMANN, Freinsheim (DE); Andreas KÜNKEL, Neustadt (DE); Radoslaw KIERAT, Altenmarkt (DE); Rolf MÜLHAUPT, Freiburg (DE); Matteo GIGLI, Freiburg (DE)

(21) Appl. No.: 15/760,428

(22) PCT Filed: Sep. 7, 2016
(86) PCT No.: PCT/EP2016/071108

§ 371 Date: Mar. 15, 2018

(30) Foreign Application Priority Data
Sep. 16, 2015 (EP) 15185503.8

(51) Int. Cl. C08G 69/28; C08G 69/26; C07C 237/06; C07C 231/02

(52) U.S. Cl. CPC C08G 69/28; C07C 231/02; C07C 237/06; C08G 69/26

(57) ABSTRACT

The present invention relates to a process for preparing an amidoamine by reacting a triacid derivative (I) with at least one amine (A), the at least one amine (A) being selected from the group consisting of diethylenetriamine and a diamine (II). The molar ratio of the triacid derivative (I) to the at least one amine (A) is in the range from 1:2 to 1:<3. The present invention further relates to the amidoamine as such and also to the use of the amidoamine of the invention as a crosslinker.

The present invention relates to a process for preparing an amidoamine by reacting a triacid derivative (I) with at least one amine (A), the at least one amine (A) being selected from the group consisting of diethylenetriamine and a diamine (II). The molar ratio of the triacid derivative (I) to the at least one amine (A) is in the range from 1:2 to 1:<3. The present invention further relates to the amidoamine as such and also to the use of the amidoamine of the invention as a crosslinker.

In polymer chemistry it is frequently necessary for polymer chains to be crosslinked in order to achieve a targeted modification to the properties of a polymer. The properties under modification include in particular the hardness, toughness, melting point, and solubility of the polymer. Crosslinking may take place during the preparation of the polymers, through the use of at least one polyfunctional monomer, but it is also possible to subject polymer chains that have already been produced to crosslinking by means of a suitable crosslinker. Crosslinkers are also referred to as hardeners. A crosslinker produces three-dimensional network structures from individual polymer chains. The prior art has described various methods for producing appropriate crosslinkers and also for using these crosslinkers to crosslink polymer chains.

M. Bähr et al., Green Chem. 2012, 14, 1447-1454 describes a process for producing oligo- and polyurethanes containing no isocyanate. These oligo- and polyurethanes are produced from cyclic carbonates based on terpenes, such as limonene, and amines, such as 1,4-butanediamine, 1,6-hexamethylenediamine, isophoronediamine, and 1,8-octamethylenediamine, for example. Crosslinked, isocyanate-free oligo- and polyurethanes may likewise be obtained by reaction with triamines. The crosslinker used for example is an amidoamine, prepared from triethyl citrate and 1,6-hexamethylenediamine or 1,12-dodecamethylenediamine. For the preparation of the trifunctional amidoamine, the triethyl citrate is reacted with the diamine in a molar ratio of 1:21.

A drawback of the process described is that the crosslinked oligo- and polyurethanes obtained are extremely brittle. Moreover, the amidoamine crosslinkers used have a relatively high melting point, and so the amidoamines can only be used as crosslinkers either at high temperatures, so that they are present in liquid form, or by dissolving them in a solvent.

M. Fleischer et al., Green Chem. 2013, 15, 934-942 likewise describe a process for producing polyurethanes without isocyanate, and the crosslinking thereof with an amidoamine. The polyurethanes are produced starting from cyclic carbonates based, for example, on pentaerythritol glycidyl ethers, which are reacted with a diamine. The amidoamine crosslinker used is prepared by reaction of triethyl citrate with hexamethylenediamine, using 7.5 equivalents of hexamethylenediamine. The product obtained in the reaction contains the amidoamine and excess hexamethylenediamine. This mixture is then used to crosslink the polyurethane.

A drawback of the process described is that curing necessarily involves the use of a mixture of hexamethylenediamine and the amidoamine. This means that both the hexamethylenediamine and the amidoamine can react with the cyclic carbonate, thus making it very difficult to control the crosslinking. If only the amidoamine is to be used in the process described, it is vital for the hexamethylenediamine to be removed beforehand, and this makes the process very inelegant and expensive.

WO 2012/171659 likewise describes the preparation of isocyanate-free polyurethanes, They are prepared starting from a terpene derivative, which contains at least two cyclic carbonate groups, and an amine. The amine used may be an amidoamine having a functionality of >2. According to WO 2012/171659, the amidoamine is prepared starting from a citric ester and a diamine selected from 1,4-diaminobutane, 1,5-diaminopentane, and 1,6-diaminohexane. The citric ester is used in a molar ratio of 1:3 relative to the amidoamine. The amidoamine is therefore a trifunctional amidoamine, thus having an amine functionality of 3.

CN 101 328 267 describes the preparation of biodegradable polyamide imides. In this process, a citrate is reacted with an aliphatic diamine, whereby the molar ratio is in the range from 1:0.2 to 1:5. Preferably, hexane diamine and butane diamine are used as diamines. A polyamide is obtained, which is then reacted to polyamide imide in a second step.

CN 101 497 695 also describes the preparation of polyamide imides. The preparation is made on the basis of citric acid esters and aliphatic diamines in a molar ratio of 1:0.1 to 1:10. Preferably, hexane diamine and butane diamine are used as diamines. A polyamide is obtained, which is subsequently reacted to polyamide imide.

A drawback of all of the above-described processes for preparing an amidoamine as crosslinker is that the amidoamines are customarily obtained in solid form. As a result they are difficult to meter and may need to be diluted for use as crosslinkers, to allow them to be metered in liquid form. If they are not to be diluted, the crosslinking must be carried out at relatively high temperatures, so that the amidoamines are in liquid form and can also be liquidly metered. A further drawback is that, owing to the amidoamines used as crosslinkers in the prior art, the resulting crosslinked polyurethanes are frequently very brittle. Moreover, the amidoamines described in the prior art have a relatively low amine functionality, which may result in poor crosslinking. Furthermore, synthesis and purification of the pure amidoamines described in the prior art are very costly and inconvenient, since a large excess of the amine component is always needed, and must be removed from the system again in the wake of successful reaction.

It was therefore an object of the present invention to provide a process for preparing amidoamines which are suitable for use as crosslinkers for polymers. The process is to be able to be carried out extremely easily and inexpensively, and the drawbacks described above for the processes described in the prior art are to occur only to a reduced extent, if at all.

This object is achieved by means of a process for preparing an amidoamine by reacting a triacid derivative of the general formula (I)

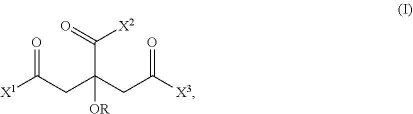

in which
X¹ is selected from the group consisting of Cl, Br, I, and OR¹;
X² is selected from the group consisting of Cl, Br, I, and OR²;
X³ is selected from the group consisting of Cl, Br, I, and OR³,
   where
R¹, R², and R³independently of one another are selected from the group consisting of hydrogen and unsubstituted or at least monosubstituted C₁-C₂₀ alkyl,
   where
   the substituents are selected from the group consisting of F, Cl, Br, OH, CN, and C₁-C₁₀ alkyl;
R is selected from the group consisting of hydrogen and C(═O)R⁴,
   where
R⁴ is an unsubstituted or at least monosubstituted C₁-C₂₀ alkyl,
   where
   the substituents are selected from the group consisting of F, Cl, Br, OH, CN, and C₁-C₁₀ alkyl;
   with at least one amine (A) selected from the group consisting of diethylenetriamine and a diamine of the general formula (II)

H2N—R⁵—NH2 (II)

in which
R⁵ is a branched C₃-C₃₁ alkyl or an unbranched Cₙ alkyl,
   where
n is an odd integer in the range from 3 to 31,
   wherein the molar ratio of the triacid derivative (I) to the at least one amine (A) is in the range from 1:2 to 1:<3.

It has surprisingly been found that with the process of the invention it is possible to prepare an amidoamine having high purity and high functionality. The amidoamine prepared in accordance with the invention also generally has no crystalline domains and possesses a low glass transition temperature T_{g}.

The glass transition temperature T_{g} is situated customarily at temperatures beneath the temperatures at which the amidoamine is metered for use as a crosslinker in a crosslinking reaction. This means that the amidoamine of the invention is normally in fluid form at the temperatures at which it is metered. This makes it very easy to meter the inventively prepared amidoamine for use in a crosslinking reaction.

The process of the invention is also quick to implement and there is no absolute need for the presence of a catalyst, thereby making the process of the invention very cost-effective.

The amidoamine of the invention can be used as a crosslinker. It possesses low toxicity, is at least partly biodegradable, and is based on renewable raw materials.

Moreover, it possesses good thermal and chemical stability, a feature likewise beneficial to its use as a crosslinker.

The process of the invention is detailed below.

### Triacid Derivative (I)

A triacid derivative (I) is reacted in accordance with the invention. For the purposes of the present invention, “a triacid derivative (I)” means either precisely one triacid derivative (I) or else a mixture of two or more triacid derivatives (I).

For the purposes of the present invention, the term “triacid derivative (I)” encompasses not only the triacid as such but also compounds derived therefrom, subject to the proviso that they can be described by the general formula (I).

In accordance with the invention the substituents in the general formula (I) have the following definitions:
X¹ is selected from the group consisting of Cl, Br, I, and OR¹;
X² is selected from the group consisting of Cl, Br, I, and OR²;
X³ is selected from the group consisting of Cl, Br, I, and OR³,
   where
R¹, R², R³ independently of one another are selected from the group consisting of hydrogen and unsubstituted or at least monosubstituted C₁-C₂₀ alkyl,
   where
   the substituents are selected from the group consisting of F, Cl, Br, OH, CN, and C₁-C₁₀ alkyl;
R is selected from the group consisting of hydrogen and C(═O)R⁴,
   where
   R⁴ is an unsubstituted or at least monosubstituted C₁-C₂₀ alkyl,
      where
      the substituents are selected from the group consisting of F, Cl, Br, OH, CN, and C₁-C₁₀ alkyl.

C(═O)R⁴ denotes an acyl group. Acyl groups are familiar to the skilled person.

In one preferred embodiment the substitutents of the triacid derivative (I) have the following definitions:
X¹ is selected from the group consisting of Cl, Br, and OR¹;
X² is selected from the group consisting of Cl, Br, and OR²;
X³ is selected from the group consisting of Cl, Br, and OR³,
   where
R¹, R², and R³ independently of one another are selected from the group consisting of hydrogen and unsubstituted or at least monosubstituted C₁-C₁₀ alkyl;
   where
   the substituents are selected from the group consisting of F, Cl, Br, OH, CN, C₁-C₅ alkyl;
R is selected from the group consisting of hydrogen and C(═O)R⁴,
   where
   R⁴ is an unsubstituted or at least monosubstituted C₁-C₁₀ alkyl,
      where
      the substituents are selected from the group consisting of F, Cl, Br, OH, CN, and C₁-C₅ alkyl.

In one especially preferred embodiment the substituents of the triacid derivative (I) have the following definitions:
X¹ is OR¹;
X² is OR²;
X³ is OR³,
   where
   R¹, R², and R³ independently of one another are selected from the group consisting of hydrogen and unsubstituted C₁-C₄ alkyl;
R is hydrogen,

Most preferably the substituents of the triacid derivative (I) have the following definitions:
X¹ is OR¹;
X² is OR²;
X³ is OR³,
   where
R¹, R², and R³are identical and are selected from the group consisting of hydrogen, methyl, and ethyl;
R is hydrogen.

If X¹ is OR¹, X² is OR², and X³ is OR³, and R¹, R², R³, and R are all hydrogen, then the triacid derivative (I) is citric acid. The IUPAC name of citric acid is 2-hydroxypropane-1,2,3-tricarboxylic acid. It carries the CAS number 77-92-9.

If X¹ is OR¹, X² is OR², and X³ is OR³, and R¹, R², and R³ are all three methyl and R is hydrogen, then the triacid derivative (I) is trimethyl citrate (citric acid trimethyl ester). The IUPAC name of trimethyl citrate is trimethyl 2-hydroxypropane-1,2,3-tricarboxylate, It carries the CAS number 1587-20-8.

If X¹ is OR¹, X² is OR², and X³ is OR³, and R¹, R², and R³ are all three ethyl and R is hydrogen, then the triacid derivative (I) is triethyl citrate (citric acid triethyl ester). The IUPAC name of triethyl citrate is triethyl 2-hydroxypropane-1,2,3-tricarboxylate. It carries the CAS number 77-93-0.

Most preferably in accordance with the invention, therefore, the triacid derivative (I) is selected from the group consisting of citric acid, trimethyl citrate, and triethyl citrate.

The present invention accordingly also provides a process wherein the triacid derivative (I) is selected from the group consisting of citric acid, trimethyl citrate, and triethyl citrate.

In another embodiment of the present invention it is possible to use citric anhydride as the triacid derivative. This anhydride is known per se to the skilled person.

Processes for preparing the triacid derivative (I) are known per se to the skilled person. If, in the triacid derivative (I), the substituent X¹ is OR¹, X² is OR², and X³ is OR³, and R¹, R², and R³ are, for example, C₁-C₂₀ alkyl, and R is hydrogen, then the triacid derivative (I) is a citric triester. This ester may be prepared, for example, by reacting citric acid with at least one alcohol. This reaction is known to the skilled person.

### Amine (A)

In accordance with the invention the triacid derivative (I) is reacted with at least one amine (A).

“At least one amine (A)” refers for the purpose of the present invention not only to precisely one amine (A) but also to a mixture of two or more amines (A).

In accordance with the invention the at least one amine (A) is selected from the group consisting of diethylenetriamine and a diamine (II). With preference in accordance with the invention the at least one amine (A) is a diamine (II).

The present invention accordingly also provides a process in which the at least one amine (A) is a diamine (II).

In accordance with the invention the substituents in the diamine (II) have the following definitions:
R⁵ is a branched C₃-C₃₁ alkyl or an unbranched Cₙ alkyl,
   where
   n is an odd integer in the range from 3 to 31

The substituents of the diamine (II) preferably have the following definitions:
R⁵ is a branched C₃-C₂₀ alkyl or an unbranched Cₙ alkyl,
   where
   n is an odd integer in the range from 3 to 2

With particular preference the substituents of the diamine (II) have the following definitions:
R⁵ is a branched C₃-C₁₃ alkyl or an unbranched Cₙ alkyl,
   where
   n is an odd integer in the range from 3 to 13.

The present invention thus also provides a process in which the amine (A) used is a diamine of the general formula (II)

in which
R⁵ is a branched C₃-C₁₃ alkyl or an unbranched Cₙ alkyl,
   where
n is an odd integer in the range from 3 to 13.

In a further embodiment of the invention, the substituents of the diamine (II) have the following definitions:
R⁵ is an unbranched Cₙ alkyl,
   where
   n is an odd integer in the range from 3 to 31, preferably in the range from 3 to 21, and especially preferably in the range from 3 to 13.

In the further embodiment the substituents of the diamine (II) have the following definitions:
R⁵ is a branched C₃-C₃₁ alkyl, preferably a branched C₃-C₂₀ alkyl, and especially preferably a branched C₃-C₁₃ alkyl.

The at least one amine (A) preferably is further selected from the group consisting of diethylenetriamine, 1,2-diaminopropane, 1,3-diaminopropane, 2,2-dimethyl-1,3-propanediamine, 1,5-diaminopentane, 1,5-diamino-2-methylpentane, 1,7-diaminoheptane, 2-butyl-2-ethyl-1,5-pentanediamine, 2,2,4-trimethyl-1,6-hexanediamine, and 2,4,4-trimethyl-1,6-hexanediamine.

The present invention therefore also provides a process in which the at least one amine (A) is selected from the group consisting of diethylenetriamine, 1,2-diaminopropane, 1,3-diaminopropane, 2,2-dimethyl-1,3-propanediamine, 1,5-diaminopentane, 1,5-diamino-2-methylpentane, 1,7-diaminoheptane, 2-butyl-2-ethyl-1,5-pentanediamine, 2,2,4-trimethyl-1,6-hexanediamine, and 2,4,4-trimethyl-1,6-hexanediamine.

In one particularly preferred embodiment the at least one amine (A) is selected from the group consisting of 1,2-diaminopropane, 1,3-diaminopropane, 2,2-dimethyl-1,3-propanediamine, 1,5-diaminopentane, 1,5-diamino-2-methylpentane, 1,7-diaminoheptane, 2-butyl-2-ethyl-1,5-pentanediamine, 2,2,4-trimethyl-1,6-hexanediamine, and 2,4,4-trimethyl-1,6-hexanediamine.

In a further embodiment of the invention the at least one amine (A) is selected from the group consisting of 1,3-diaminopropane, 1,5-diaminopentane, and 1,7-diaminoheptane.

In a further embodiment the at least one amine (A) is selected from the group consisting of 1,2-diaminopropane, 2,2-dimethyl-1,3-dipropanediamine, 1,5-diamino-2-methylpentane, 2-butyl-2-ethyl-1,5-pentanediamine, 2,2,4-trimethyl-1,6-hexanediamine, and 2,4,4-trimethyl-1,6-hexanediamine.

By “unsubstituted or at least monosubstituted C₁-C₂₀ alkyl” is meant, for the purposes of the present invention, saturated and unsaturated hydrocarbons having a free valence (radical) and from 1 to 20 carbon atoms. The hydrocarbons may be linear or cyclic. It is also possible for them to include a cyclic and a linear component. Where the C₁-C₂₀ alkyls are at least monosubstituted, they may additionally contain branching in the form of C₁-C₁₀ alkyl groups, or other functional groups. Examples of alkyl groups are methyl, ethyl, propyl, isopropyl, n-butyl, isobutyl, sec-butyl, tert-butyl, hexyl, and cyclohexyl. Corresponding comments apply to “unsubstituted or at least monosubstituted C₁-C₁₀ alkyl” and “unsubstituted or at least monosubstituted C₁-C₅ alkyl”.

“Branched C₃-C₃₁ alkyl” refers for the purposes of the present invention to a saturated or unsaturated hydrocarbon having a free valence (radical) and from 3 to 31 carbon atoms that has at least one branch, i.e., at least one alkyl group as substituent. The number of carbon atoms refers to the total number of carbon atoms in the C₃-C₃₁ alkyl, in other words to the sum of the carbon atoms in the hydrocarbon and the at least one alkyl group as substituent. Apart from the at least one further alkyl group, the branched C₃-C₃₁ alkyl preferably has no other substituents. It therefore preferably has no substituents other than an alkyl group. Corresponding comments apply to “branched C₃-C₂₀ alkyl” and “branched C₃-C₁₃ alkyl”.

“unbranched Cₙ alkyl” for the purposes of the present invention refers to a saturated or unsaturated hydrocarbon having a free valence (radical) and n carbon atoms that has no branching, i.e., no alkyl group as substituent. The unbranched Cₙ alkyl preferably also has no substituents other than an alkyl group. An unbranched Cₙ alkyl for the purposes of the present invention is therefore preferably unsubstituted.

An “odd integer” is an integer which cannot be divided by 2 without a remainder, Examples thereof are 3, 5, 7, 9, 11, 13, 15, 17, 19, 21, 23, 25, 27, 29, and 31.

### Reaction

In accordance with the invention the triacid derivative (I) is reacted with the at least one amine (A) for preparing the amidoamine.

The reaction may take place according to any methods known to the skilled person. The reaction of the triacid derivative (I) with the at least one amine (A) may be carried out, for example, in the presence of a solvent. It is also possible for the reaction to take place without a solvent. With preference the reaction of the triacid derivative (I) with the at least one amine (A) takes place in the absence of a solvent.

It is possible, furthermore, for the reaction of the triacid derivative (I) with the at least one amine (A) to take place in the presence of a catalyst which catalyzes the reaction of the triacid derivative (I) with the at least one amine (A). The reaction preferably takes place in the absence of a catalyst.

The reaction of the triacid derivative (I) with the at least one amine (A) may take place in any form of reactor known to the skilled person. Examples of suitable reactors are stirred tank reactors, stirred tank cascades, or tubular reactors.

The reaction of the triacid derivative (I) with the at least one amine (A) may take place at any desired temperature. Preferably the reaction of the triacid derivative (I) with the at least one amine (A) takes place at a temperature T in the range from 20 to 200° C. More preferably the reaction takes place at a temperature in the range from 40 to 120° C., and especially preferably at a temperature in the range from 50 to 100° C.

The present invention therefore also provides a process in which the reaction of the triacid derivative (I) with the at least one amine (A) takes place at a temperature T in the range from 20 to 200° C.

The pressure during the reaction of the triacid derivative (I) with the at least one amine (A) may be any desired pressure. For example, the pressure during the reaction of the triacid derivative (I) with the at least one amine (A) may be in the range from 0.1 mbar to 10 bar, preferably in the range from 1 mbar to 2 bar, and especially preferably in the range from 750 mbar to 1.5 bar.

The reaction of the triacid derivative (I) with the at least one amine (A) may be carried out for any desired time. The time for the reaction of the triacid derivative (I) with the at least one amine (A) is situated, for example, in the range from 0.5 to 18 hours, preferably in the range from 1 to 10 hours, and especially preferably in the range from 2 to 8 hours.

For the reaction of the triacid derivative (I) with the at least one amine (A), the triacid derivative (I) may be introduced as an initial charge to the reactor, then brought to the temperature T at which the reaction takes place, and subsequently the at least one amine (A) may be added. It is also possible first to introduce the at least one amine (A) as an initial charge to the reactor and to bring it to a temperature T at which the reaction takes place, and subsequently to add the triacid derivative (I). A further possibility is to introduce the triacid derivative (I) and the at least one amine (A) as a joint initial charge to a reactor, optionally with stirring, and then to bring this initial charge to the temperature T at which the reaction takes place. With preference the triacid derivative (I) and the at least one amine (A) are charged jointly to a reactor and are brought with stirring to the temperature T at which the reaction takes place.

The reaction during the reaction of the triacid derivative (I) with the at least one amine (A) is known as such to the skilled person.

During the reaction of the triacid derivative (I) with the at least one amine (A), one of the amino groups (NH₂ group) of the at least one amine (A) reacts with one of the COX¹, COX² or COX³ groups of the triacid derivative (I) to give an amide group. In this process, X¹H, X²H or X³H is eliminated.

This means, if the group with which the amino group of the at least one amine (A) reacts is an ester group, for example, it is an alcohol that is eliminated. If the group with which the amino group of the at least one amine (A) reacts is a carboxylic acid group, then water is eliminated.

The alcohol or the water is customarily removed after or during the reaction of the triacid derivative (I) with the at least one amine (A). The alcohol or the water may be removed by any methods known to the skilled person, as for example by distillation or by addition of a drying agent.

With preference the water or the alcohol is removed by distillation, more preferably by distillation under reduced pressure.

The molar ratio of the triacid derivative (I) to the at least one amine (A) is situated in accordance with the invention in the range from 1:2 to 1:<3; preferably the molar ratio of the triacid derivative (I) to the at least one amine (A) is situated in the range from 1:2.4 to 1:2.8.

The present invention therefore also provides a process in which the molar ratio of the triacid derivative (I) to the at least one amine (A) is in the range from 1:2.4 to 1:2.8.

It will be readily appreciated that the molar ratio of the triacid derivative (I) to the at least one amine (A) refers to the molar ratio of the triacid derivative (I) to the at least one amine (A) before the reaction, in other words before the triacid derivative (I) has reacted with the at least one amine (A).

### Amidoamine

In the reaction of the triacid derivative (I) with the at least one amine (A), in accordance with the invention, the amidoamine is formed.

During the reaction, the triacid derivative (I) reacts with the at least one amine (A) to give the amidoamine. The amidoamine therefore comprises the triacid derivative (I) and the at least one amine (A) in reacted form. The amidoamine hence contains structural units derived from the triacid derivative (I) and structural units derived from the at least one amine (A).

The amidoamine is obtained preferably as an oligomer in the reaction. Oligomers are formed when an amino group (NH₂ group) of the at least one amine (A) reacts with a triacid derivative (I) and subsequently a second amino group of the at least one amine (A) reacts with a second triacid derivative (I), which may in turn react with a further at least one amine (A).

For the purposes of the present invention, an oligomer of the amidoamine means that the amidoamine contains in the range from 3 to 280 structural units derived from the at least one amine (A), and in the range from 1 to 120 structural units derived from the triacid derivative (I).

An oligomer of the amidoamine preferably contains in the range from >3 to 140 structural units derived from the at least one amine (A), and in the range from 1 to 60 structural units derived from the triacid derivative (I).

An oligomer of the amidoamine more preferably contains in the range from 4 to 15 structural units derived from the at least one amine (A), and in the range from 1 to 6 structural units derived from the triacid derivative (I).

An oligomer of the amidoamine especially preferably contains in the range from 4 to 12 structural units derived from the at least one amine (A), and in the range from 1 to 5 structural units derived from the triacid derivative W.

The amidoamine prepared in accordance with the invention preferably contains no crystalline constituents. The amidoamine is therefore preferably amorphous. This means that the amidoamine of the invention preferably has no melting temperature. The glass transition temperature T_{g} of the amidoamine of the invention is preferably no more than room temperature (20° C.). For example, the glass transition temperature T_{g} of the amidoamine is in the range from −40 to 20° C.

The present invention therefore also provides a process in which the amidoamine has a glass transition temperature T_{g} in the range from −40 to 20° C.

The amidoamine of the invention is therefore preferably fluid at room temperature (20° C.). “Fluid” in the context of the present invention means that the amidoamine has a glass transition temperature T_{G} at or below room temperature (20° C.) and/or is pumpable by means of conventional pumps at temperatures between 20° C. and 80° C. The terms “liquid” and “fluid” are used synonymously in the context of the present invention. They therefore possess the same meaning.

The amidoamine of the invention therefore customarily has a viscosity in the range from 1000 to 1 000 000 mPas, preferably in the range from 1000 to 200 000 mPas, and especially preferably in the range from 1000 to 100 000 mPas, measured at 60° C. with an Anton Paar Physica MCR 301 rheometer with plate/plate geometry, with a shear rate of 1/s, 6 sec/data point, 20 data points, 1 mm gap width.

The present invention accordingly also provides a process in which the amidoamine has a viscosity in the range from 1000 to 1 000 000 mPas as measured at 60° C. with an Anton Paar Physica MCR 301 rheometer with plate/plate geometry, shear rate 1/s, 6 sec/data point, 20 data points, 1 mm gap width.

The amidoamine of the invention preferably has a functionality in the range from 3 to 40, more preferably in the range from 3 to 21, and especially preferably in the range from 3 to 11.

The amidoamine of the invention has, for example, a number-average molecular weight Mₙ in the range from 500 to 30 000 g/mol, preferably in the range from 800 to 20 000 g/mol, and especially preferably in the range from 1000 to 15 000 g/mol, as determined by gel permeation chromatography (GPC) using a Waters Alliance 2695 separation module with Shodex OHpak SB-804HQ, SB-802,5HQ (300×8.0 mm) column and 0.3 mol/L sodium acetate, pH 4.5 (adjusted using acetic acid) as eluent, flow rate: 0.5 mL/min; injection: 50 μL, detector: Waters Refractive Index (RI) 2410, calibration: pullulan or PEG/PEO.

The present invention hence also provides a process in which the amidoamine has a weight-average molecular weight M_{w} in the range from 500 to 30 000 g/mol.

Furthermore, the present invention provides a process in which the amidoamine has a number-average molecular weight Mₙ in the range from 500 to 30 000 g/mol.

The polydispersity of the amidoamine of the invention is situated for example in the range from 1.1 to 20, preferably in the range from 1.3 to 10, and especially preferably in the range from 1.5 to 5. Polydispersity refers to the ratio of the weight-average molecular weight M_{w} to the number-average molecular weight Mₙ.

The present invention also provides an amidoamine obtainable by the process of the invention.

The amidoamine of the invention may be used, for example, as a crosslinker, in the preparation of polyaddition or polycondensation polymers, for example.

The present invention hence also provides for the use of the amidoamine of the invention as a crosslinker.

The amidoamine of the invention can be used as a crosslinker in all reactions known to the skilled person for which amines as crosslinkers are suitable.

The amidoamine of the invention is used preferably as a crosslinker for thermosettingly curable resin systems.

The present invention hence also provides for the use of the amidoamine as a crosslinker for thermosettingly curable resin systems.

Suitable thermosetting curable resin systems are known per se to the skilled person. For example, thermosetting curable resin systems are selected from the group consisting of thermosettingly curable isocyanate resin systems, thermosettingly curable urethane resin systems, thermosettingly curable epoxy resin systems, thermosettingly curable polyester resin systems, thermosettingly curable polyamide resin systems, and thermosettingly curable carbonate resin systems.

The present invention hence also provides for the use of the amidoamine of the invention as a crosslinker, where the thermosettingly curable resin systems are selected from the group consisting of thermosettingly curable isocyanate resin systems, thermosettingly curable urethane resin systems, thermosettingly curable epoxy resin systems, thermosettingly curable polyester resin systems, thermosettingly curable polyamide resin systems, and thermosettingly curable carbonate resin systems.

The aforementioned thermosettingly curable resin systems are known per se to the skilled person.

The present invention is elucidated in more detail below by examples, but without being confined to these examples.

### EXAMPLES

Triacid derivative (I) used with triethyl citrate (citric acid triethyl ester, ≥99%, FCC, Sigma Aldrich).

Amines used as amine (A) were as follows:
1,5-diaminopentane (pentamethylenediamine, PMDA)
1,3-diaminopropane (trimethylenediamine, TMDA)
diethylenetriamine (DETA)
2,2 ,4-trimethyl-1,6-hexanediamine (TMHDA)

In examples C1 and 2 to 7, the triacid derivative (I) was prepared with the amine (A) indicated in Table 1a, in the molar ratio indicated in Table 1a (triacid derivative (I) to amine (A)), in a glass flask equipped with stirrer and reflux condenser, and this mixture was heated to the temperature specified in Table 1a, and held at this temperature for the reaction time indicated in Table 1a. Thereafter the reaction mixture obtained was transferred while still hot into a single-neck flask, where it was freed from ethanol formed in the reaction and optionally from residual monomeric amine (A) on a rotary evaporator at 55° C. under a pressure of 1 mbar for a period of 30 minutes.

In example 8, the triacid derivative (I) was prepared with the amine (A) indicated in Table 1a, in the molar ratio indicated in Table 1a (triacid derivative (I) to amine (A)), in a glass flask equipped with stirrer, descending condenser, and catch vessel, and this mixture was heated to the temperature indicated in Table 1a and held at this temperature for the reaction time indicated in Table 1a. Ethanol formed during the reaction was removed continuously from the reaction mixture. After the end of the reaction, the mixture was transferred while still hot into a single-neck flask, where residual ethanol and any remaining monomeric amine (A) were removed on a rotary evaporator at 55° C. under a pressure of 1 mbar over a time of 30 minutes.

In Tables 1a and 1b, the following parameters and results for the examples are indicated:
Molar ratio: Molar ratio of triacid derivative (I) to amine (A).
Temperature: Temperature at which the reaction was carried out.
Reaction time: Time for which the reaction was carried out
η Viscosity of the resulting amidoamine, determined at 60° C. using an Anton Paar Physica MCR 301 rheometer with plate/plate geometry, shear rate 1/s, 6 s/data point, 20 data points, 1 mm gap width.
M_{w} and Mₙ: Weight-average and number-average molecular weight, determined by gel permeation chromatography (GPC) using a Waters Alliance 2695 separation module with Shodex OHpak SB-804HQ, SB-802.5HQ (300×8.0 mm) column and 0.3 mol/L sodium acetate, pH 4.5 (adjusted using acetic acid) as eluent. (Flow rate: 0.5 mL/min; injection: 50 μL, detector: Waters Refractive Index (RI) 2410, calibration: PEG/PEO).
T_{g}: The glass transition temperatures (T_{g}) were measured using a DSC-7 heat flow calorimeter from Perkin-Elmer. For this purpose, 5 to 7 mg of the sample were weighed out into an aluminum crucible, and measurement took place in a temperature range from −100 to +100° C. with a heating and cooling rate of 10 K min⁻¹. The glass transition temperatures (T_{g} (I) and T_{g} (II)) were determined from the first and second heating curves, respectively.

TABLE 1a

| | | | Reaction time | Temperature |
|---|---|---|---|---|
| Example | Amine (A) | Molar ratio | [h] | [° C.] |
| C1 | PMDA | 1:3.0 | 4 | 90 |
| 2 | PMDA | 1:2.8 | 4 | 90 |
| 3 | PMDA | 1:2.6 | 4 | 90 |
| 4 | PMDA | 1:2.3 | 4 | 80 |
| 5 | TMHDA | 1:2.6 | 5.5 | 90 |
| 6 | DETA | 1:2.6 | 6 | 80 |
| 7 | TMDA | 1:2.6 | 5 | 80 |
| 8 | PMDA | 1:2.8 | 2 | 100 |

TABLE 1b

| | Mₙ | M_{w} | η | T_{g} (I) | T_{g} (II) |
|---|---|---|---|---|---|
| Example | [g/mol] | [g/mol] | [mPas] | [° C.] | [° C.] |
| C1 | 1200 | 3600 | 102 600 | −14 | 8 |
| 2 | 11 400 | 33 500 | 22 300 | −35 | −4 |
| 3 | 14 200 | 88 200 | 46 800 | −29 | −1 |
| 4 | 12 300 | 102 000 | 53 700 | −23 | 2 |
| 5 | 14 900 | 37 200 | 79 800 | −44 | −25 |
| 6 | 6400 | 14 200 | 20 800 | −43 | −7 |
| 7 | 9700 | 34 600 | 48 400 | −26 | 5 |
| 8 | 12 300 | 36 800 | 23 200 | −32 | −6 |

1.-13. (canceled)

14. A process for preparing an amidoamine comprising reacting a triacid derivative of the general formula (I) 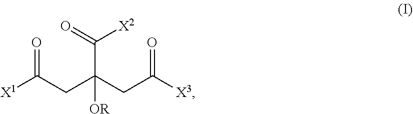
in which
X¹ is selected from the group consisting of Cl, Br, I, and OR¹;
X² is selected from the group consisting of Cl, Br, I, and OR²;
X³ is selected from the group consisting of Cl, Br, I, and OR³,
where
R¹, R², and R³ independently of one another are selected from the group consisting of hydrogen and unsubstituted or at least monosubstituted. C₁-C₂₀ alkyl,
where
the substituents are selected from the group consisting of F, Cl, Br, OH, CN, and C₁-C₂₀ alkyl;
R is selected from the group consisting of hydrogen and C(═O)R⁴,
where
R⁴ is an unsubstituted or at least monosubstituted C₁-C₂₀ alkyl,
where
the substituents are selected from the group consisting of F, Cl, Br, OH, CN, and C₁-C₁₀ alkyl;
with at least one amine (A) selected from the group consisting of diethylenetriamine and a diamine of the general formula (II)
H₂N—R⁵—NH₂ (II)
in which
R⁵ is a branched C₃-C₃₁ alkyl or an unbranched Cₙ alkyl,
where
n is an odd integer in the range from 3 to 31,
wherein the molar ratio of the triacid derivative (I) to the at least one amine (A) is in the range from 1:2 to 1:<3.

15. The process according to claim 14, wherein the reaction of the triacid derivative (I) with the at least one amine (A) takes place at a temperature T in the range from 20 to 200° C.

16. The process according to claim 14, wherein the molar ratio of the triacid derivative (I) to the at least one amine (A) is in the range from 1:2.4 to 1:2.8.

17. The process according to claim 14, wherein the triacid derivative (I) is selected from the group consisting of citric acid, trimethyl citrate, and triethyl citrate.

18. The process according to claim 14, wherein the amine (A) is a diamine of the general formula (II)
in which
R⁵ is a branched C₃-C₁₃ alkyl or an unbranched Cₙ alkyl,
where
n is an odd integer in the range from 3 to 13.

19. The process according to claim 14, wherein the at least one amine (A) is selected from the group consisting of diethylenetriamine, 1,2-diaminopropane, 1,3-diaminopropane, 2,2-dimethyl-1,3-propanediamine, 1,5-diaminopentane, 1,5-diamine-2-methylpentane, 1,7-diaminoheptane, 2-butyl-2-ethyl-1,5-pentanediamine, 2,2,4-trimethyl-1,6-hexanediamine, and 2,4,4-trirnethyl-1,6-hexanediamine.

20. The process according to claim 14, wherein the amidoamine has a number-average molecular weight Mₙ in the range from 500 to 30 000 g/mol.

21. The process according to claim 14, wherein the amidoamine has a viscosity in the range from 1000 to 1 000 000 mPas as measured at 60° C. using an Anton Paar Physica MCR 301 rheometer with plate/plate geometry, shear rate 1/s, 6 sec/data point, 20 data points, 1 mm gap width.

22. The process according to claim 14, wherein the amidoamine has a glass transition temperature T_{g} in the range from −40 to 20° C.

23. An amidoamine obtained by the process according to claim 14.

24. The use of the amidoamine according to claim 23 as a crosslinker.

25. A thermosettingly curable resin system comprising the amidoamine according to claim 23 as a crosslinker.

26. The thermosettingly curable resin system according to claim 25, wherein the thermosettingly curable resin system is selected from the group consisting of thermosettingly curable isocyanate resin systems, thermosettingly curable urethane resin systems, thermosettingly curable epoxy resin systems, thermosettingly curable polyester resin systems, thermosettingly curable polyamide resin systems, and thermosettingly curable carbonate resin systems.
